(19) United States
(12) Patent Application Publication
(10) Pub. No.: US 2026/0102567 A1
(43) Pub. Date: Apr. 16, 2026

(54) INJECTION SYRINGE

(71) Applicant: DOMEJECT AB, Rejmyre (SE)

(72) Inventor: Billy NILSSON, Mjölby (SE)

(21) Appl. No.: 19/136,123

(22) PCT Filed: Dec. 8, 2023
(86) PCT No.: PCT/SE2023/051237

§ 371 Date: Jun. 5, 2025

(30) Foreign Application Priority Data
Dec. 8, 2022 (SE) 2230400-0

(51) Int. Cl. A61M 5/24; A61M 5/315

(52) U.S. Cl. CPC A61M 5/2425; A61M 5/31513; A61M 2005/31516

(57) ABSTRACT

Injection syringe for injecting medication or sampling, where only part of the syringe is discarded with each use while the rest of the syringe is used repeatedly. Injection syringe (1) comprises a barrel (2), barrel seal (3), plunger (4), plunger packing (5), which can be moved back and forth therein, and a compressible ellipsoid capsule (6), which is removably attached to the outlet end of the barrel. The barrel chamber is adapted to the ellipsoidal capsule. The capsule can be compressed with the plunger. The barrel volume between the plunger displacement positions, which corresponds to the empty and the full capsule, is essentially equal to the volume of the capsule.

### FIELD OF THE INVENTION

This invention relates to improvements in injection syringes for subcutaneous, intravenous, intramuscular and rectal injection (or other forms of injection where the syringe can conveniently be used) of drugs and similar solutions in humans or animals, as well as syringes for taking blood samples from them.

More specifically, the syringe according to the invention is of the type where only a part of the syringe is discarded at each use, while the rest of the syringe is used repeatedly. Even more specifically, the invention relates to a syringe comprising an ellipsoidal capsule and a cylindrical barrel that is adapted to it.

### DESCRIPTION OF THE STATE OF THE ART

It is well known that most injection syringes now used in hospitals, doctor's offices and health centers are of the disposable type, i.e., they are thrown away or discarded after a single use. These injection syringes usually comprise three parts: a barrel, a plunger and a seal or, more commonly, two seals, which are pre-assembled for reciprocating movement in the barrel, wherein the first two parts being usually made of plastic material and the seal of flexible material.

As a disposable product, an injection injection syringe of this type, even if manufactured by modern mass production processes such as injection molding, is very inexpensive due to the materials and precision manufacturing required.

The present inventor has previously proposed to combine a barrel and a plunger with a replaceable capsule, which is placed in the outlet end of the barrel, where the capsule is compressed against itself during use by the action of the plunger when extruding or dispensing the medicine contained in the capsule. The stroke volume of the plunger is related to the volume of the capsule to eliminate the occurrence of a significant negative pressure when the plunger is retracted to fill the capsule, and the plunger can conform itself to the shape of the capsule for complete emptying of the capsule. A syringe of this type is shown in WO 81/02838.

A disadvantage of this syringe is that it can be difficult to dose the force to be applied to the plunger's handle when injecting the liquid, so that the plunger only compresses the capsule and liquid is injected and does not release the capsule at the same time. Another disadvantage is that the plunger packing, when returning the plunger and suction of liquid, does not hold tight and causes air to enter the syringe, which means that the volume of suctioned liquid cannot be determined and the procedure must be redone. Another disadvantage is that it is difficult to dose the exact volume of the medicine as the volumes that are sometimes handled are very small and the distance between the scale lines of the syringe is very small.

### SUMMARY OF THE INVENTION

The main purpose of the present invention is to solve the above-mentioned problems and disadvantages of the previously known injection syringe with disposable capsule by providing a injection syringe in which the dosage of the medicinal liquid is more accurate and easier, and the release of the disposable capsule is easier and safer.

This is achieved mainly by the fact that the capsule is essentially ellipsoidal and that the barrel chamber of the syringe is adapted to this. For the purposes of the invention, the shape of the capsule is preferably free of spherical or straight cylindrical wall sections, and ranges from ellipsoidal to more narrow ellipsoidal shape (olive-shaped).

Further purposes and advantages of the invention will partly be highlighted in the description that follows and partly apparent from the description or can be experienced by practicing the invention. The purposes and advantages of the invention can be realized and achieved through the devices and combinations which are particularly emphasized in the appended patent claims.

In order to achieve the above-related purposes, according to the invention there has been provided a injection syringe of the kind and with the characteristics that appear in patent claim 1.

The syringe may be provided as several separate parts which are brought together and assembled, e.g., the syringe body and the capsule. The syringe can also be provided as a kit comprising one or more of the syringe's parts, and where the syringe is unassembled or fully or partially preassembled.

Attached drawings, which are included in and form part of this description, shows an embodiment of the invention and serves together with the description to explain the principles of the invention.

### BRIEF DESCRIPTION OF THE DRAWINGS

FIG. 1A in an axial cross-sectional view of a first embodiment of the injection syringe constructed according to the invention, shown with an empty disposable capsule attached to the barrel, and with the plunger maximally extended and plunger packing fitted, where the proximal part of the plunger rod longitudinal ribs,

FIG. 1B in an axial cross-sectional view of a second embodiment of the injection syringe constructed according to the invention, shown with an empty disposable capsule attached to the barrel, and with the plunger fully extended and plunger packing fitted, and where a measuring scale is mounted inside the cylindrical and hollow plunger rod.

FIG. 2A shows the injection syringe barrel (according to the first the embodiment) in axial cross-sectional view, cross-sectional view from below, and side view.

FIG. 2B shows the injection syringe barrel (according to the second embodiment) in axial cross-sectional view, cross-sectional view from below, and side view.

FIG. 3 shows the barrel top seal in side view and proximal view, as well as axial cross-sectional view (the thicker arrows show the discharge direction).

FIG. 4A shows the plunger of the injection syringe (according to the first embodiment) in axial cross-sectional view, as well as distal view and side view.

FIG. 4B shows the plunger of the injection syringe (according to the second embodiment) in axial cross-sectional view, as well as distal view and side view.

FIG. 5 shows the separate cylindrical measuring scale (according to the second embodiment) in axial cross-sectional view, as well as distal view and side view.

FIG. 6 shows the separate cylindrical measuring scale according to FIG. 5 inserted in the plunger (according to the second embodiment) in axial cross-sectional view and distal view.

FIG. 7A show the injection syringe plunger packing with superimposed ribs, in axial cross-sectional view, as well as distal view and side view.

FIG. 7B show the injection syringe plunger packing without superimposed ribs, in axial cross-sectional view, as well as distal view and side view.

FIG. 8A shows the injection syringe's associated capsule (which matches the plunger packing in FIGS. 7A) in axial cross-sectional view, as well as proximal and side views, where the capsule is not expanded (the expanded and collapsed second half of the capsule is shown in FIG. 9A).

FIG. 8B shows the injection syringe's associated capsule (which matches the plunger packing of FIGS. 7B) in axial cross-sectional view, as well as proximal and side views, where the capsule is not expanded (the expanded and retracted second half of the capsule is shown in FIG. 9B).

FIG. 9A shows the injection syringe's associated capsule (which matches the plunger packing of FIGS. 7A) in an axial cross-sectional view and a side view, where the capsule is expanded and filled with liquid, and a larger-scale axial cross-sectional section of the distal end.

FIG. 9B shows the injection syringe's associated capsule (matching the plunger packing of FIGS. 7B) in an axial cross-sectional view and a side view, where the capsule is expanded and filled with fluid (fluid not shown), and a larger scale axial cross-sectional section of the distal end, an axial cross-sectional section larger scale of the distal end, and a larger scale axial cross-sectional view of a preferred variant of the joint of the capsule.

FIG. 10A shows an axial cross-sectional view and a side view of the injection syringe of FIGS. 1A, shown at a point in the operation sequence where a pre-filled capsule is about to be mounted in the discharge end of the barrel.

FIG. 10B shows an axial cross-sectional view and a side view of the injection syringe of FIGS. 1B, shown at a point in the operation sequence where a pre-filled capsule is about to be mounted in the discharge end of the barrel.

FIG. 11A shows views similar to FIG. 10A of the injection syringe, shown at one point in the sequence of operations where the capsule is mounted and the plunger is advanced to expel the liquid contained in the capsule.

FIG. 12B shows views similar to FIG. 10B of the injection syringe, shown at one point in the sequence of operations where the capsule is mounted and the plunger is advanced to expel the liquid contained in the capsule.

FIG. 12 shows the same view as FIG. 10A of the injection syringe, but also showing a larger scale axial cross-sectional section of the outlet end of the barrel and the attachment of the capsule therein.

FIG. 13 shows views similar to FIG. 10A of the injection syringe, shown at a point in the operation sequence where the liquid contained in the capsule has been completely expelled.

FIG. 14 shows views similar to FIG. 10A of the injection syringe, shown in the final moment of the operation sequence where the emptied capsule is ejected from the barrel.

### DETAILED DESCRIPTION

Reference is now made in detail to those currently preferred embodiments of the invention, where examples of these are shown in the attached drawings.

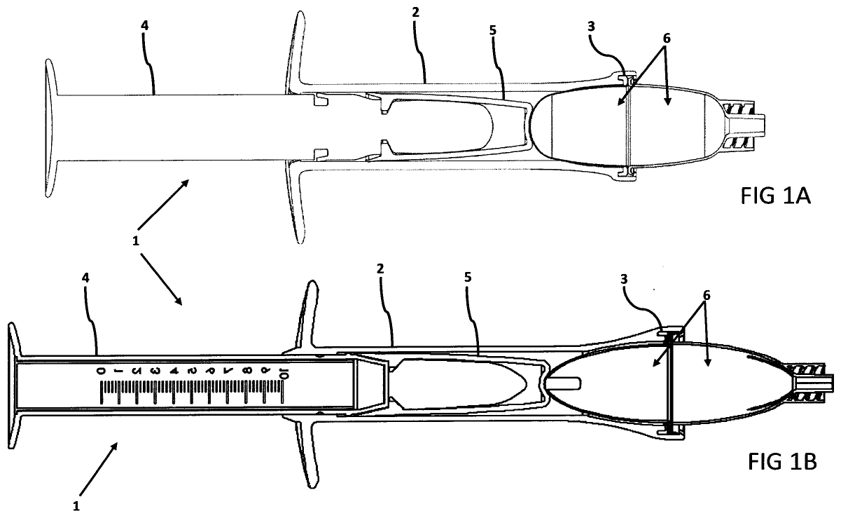

Referring to the drawings, first to FIG. 1A which shows in detail the construction of a first embodiment of a injection syringe 1 according to the invention, this injection syringe constitutes a unit in two parts, where two are designed for repeated use and comprise an elongated barrel 2 (with a barrel seal 3) with an outlet end and a plunger 4 (with a plunger packing 5), which is mounted for reciprocating movement in the barrel. This injection syringe is adapted for use with a disposable capsule 6 (which can be pre-filled or filled after assembly), which is mounted in the outlet end of the barrel. This barrel can be designed as an element in a piece of plastic material, preferably made of a transparent plastic material. FIG. 1B is a second embodiment of this injection injection syringe, with the main difference that the plunger 4 is cylindrical, hollow and transparent to be able to receive a measuring scale, and is intended to be easily removed from the barrel during for example washing.

### Barrel

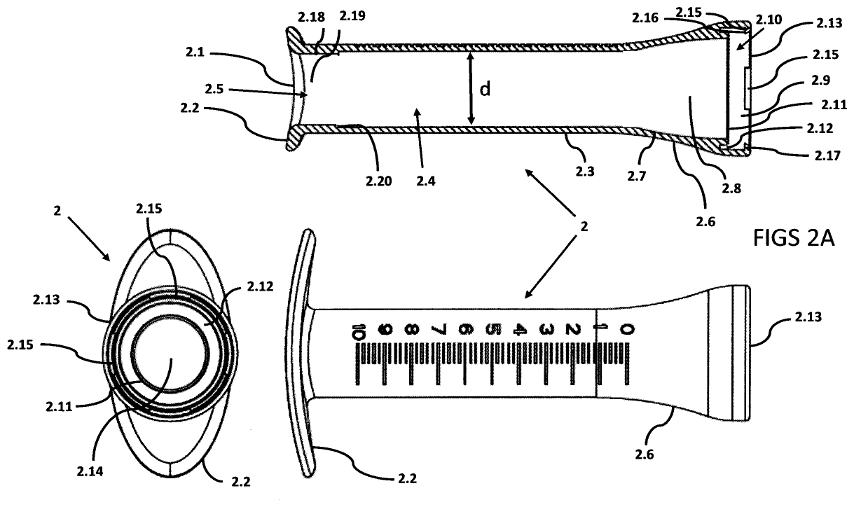

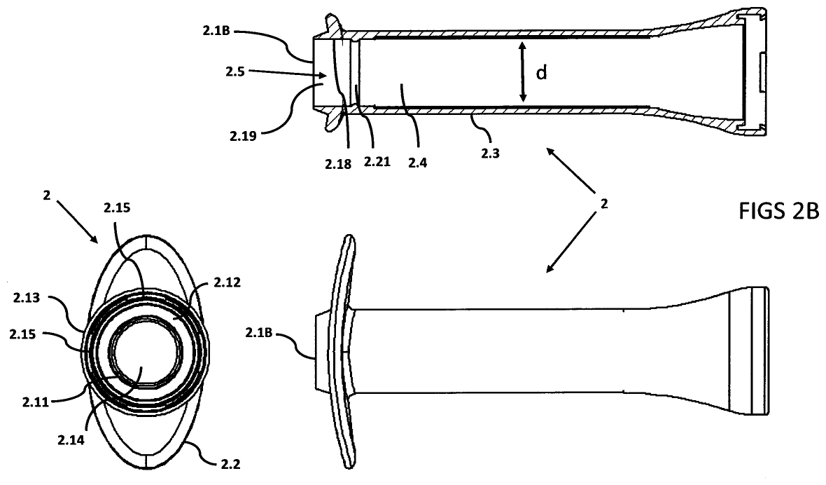

In FIGS. 2A, a first embodiment of the barrel 2 is shown, which at a proximal end 2.1, which is opposite the outlet end, has a grip part 2.2, preferably with a concave or convex shape (the alternatives are shown in several places in the drawings), which forms a thumb rest, by which the barrel can be grabbed by when handling the injection syringe. A first cylindrical wall 2.3 houses a first cylindrical cavity 2.4 (with an internal diameter d), which at its proximal end forms a proximal mouth 2.5 and at its distal end widens into a substantially cone-shaped flare 2.6 with an inner semi-ellipsoidal inner wall 2.7 enclosing a semi-ellipsoidal cavity 2.8. This cone-shaped flare 2.6 is limited at its periphery by a cylindrical part made in one piece with the barrel with an inner second cylindrical wall 2.9 which encloses a second cylindrical cavity 2.10. This wall is defined by an inner circular edge 2.11, in which there is arranged a groove 2.12, intended for receiving a seal, and an outer circular edge 2.13 which also defines a distal outlet mouth 2.14. At the outer edge 2.13 of the second cylindrical wall 2.9, peripheral mutually spaced inwardly directed strips 2.15 (e.g., 2 to 4 pieces) are formed on the inner surface, each of which extends over mainly 30°. Each strip forms an inward facing shoulder 2.16 and tapers from this shoulder towards the free edge of the second cylindrical wall 2.9 to form a conical guide surface 2.17. The guide surface is arranged to guide the capsule when attaching it to the barrel, and the pressure of the barrel seal against the capsule leads to the capsule in turn pressing against the shoulder 2.16 on the strips 2.15, which means that the capsule is held in place in the barrel. The first cylindrical wall 2.3 merges at its proximal end into a third cylindrical wall 2.18, which runs all the way towards the proximal mouth 2.5 and which has a smaller diameter. This wall 2.18 houses a third cylindrical cavity 2.19. Furthermore, this wall, through its smaller diameter, forms a proximal stop heel or surface 2.20 in the first cylindrical cavity 2.4.

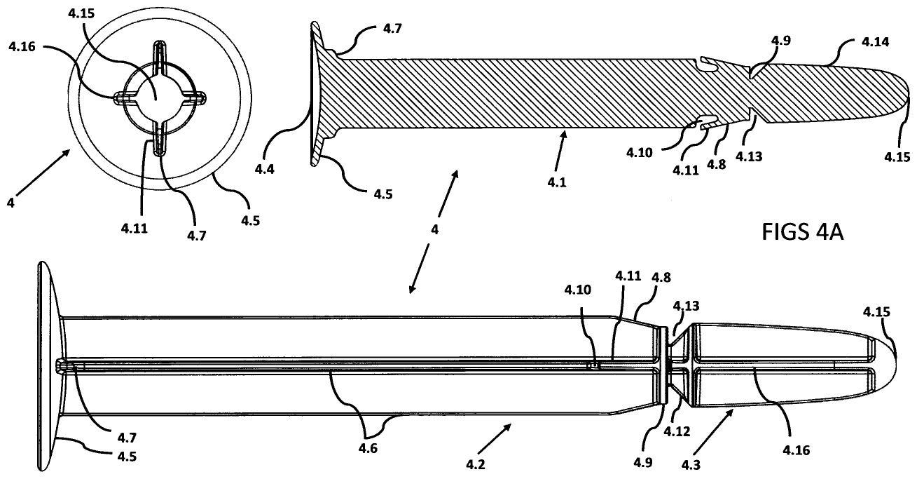

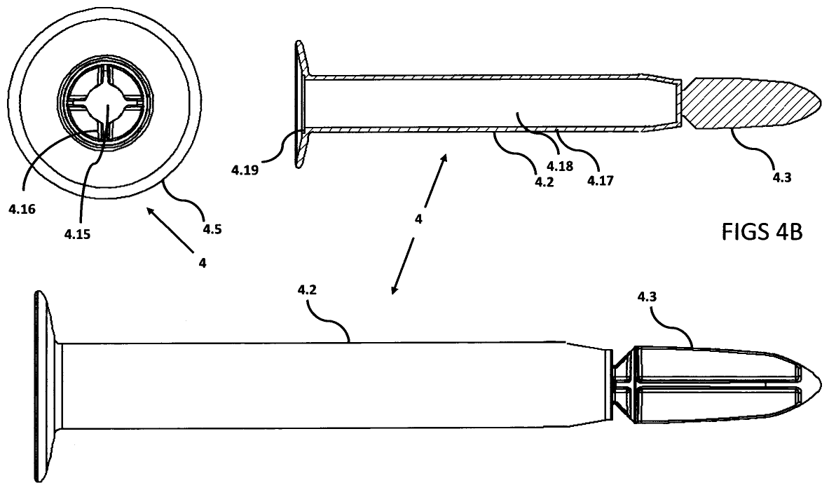

In FIGS. 2B, a barrel belonging to the second embodiment of the injection injection syringe 1 is shown, which fits together with the embodiment of the plunger 4 shown in FIG. 4B. It is essentially identical to the first embodiment, but differs in a few points. The first cylindrical wall 2.3 merges at its proximal end into a third cylindrical wall 2.18, which has the same diameter as the first cylindrical wall 2.3, and runs all the way towards the proximal mouth 2.5. This wall 2.18 houses a third cylindrical cavity 2.19. The wall 2.18 has an inwardly projecting peripheral ridge 2.21 which replaces the stop heel 2.20 in the first embodiment. This ridge 2.21 forms a soft stop when a plunger according to embodiment 2 is pulled out of the barrel. Furthermore, the proximal end 2.1 has been replaced by a projecting edge 2.1B. In one embodiment, this protruding edge is matte, opaque or less transparent than the rest of the barrel in order to be able to function as a measuring edge when a measuring scale sits on the plunger 4 or the plunger insert 7 (gives a better reading).

The cylindrical cavities, when taken together, are referred to as the cylindrical channel.

The barrel is suitably made of plastic material such as polycarbonate or polyamide.

### Barrel Seal

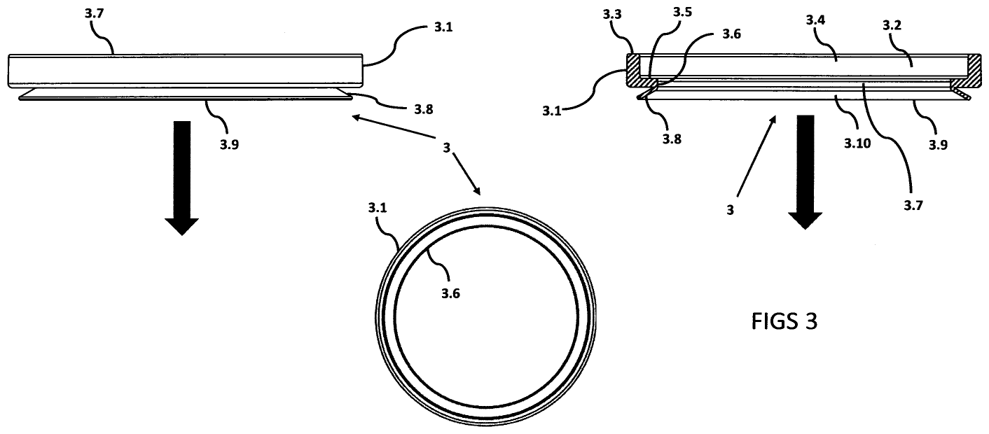

In FIGS. 3, a barrel seal 3 is shown which will be described based on how it will be mounted in the outlet end of the barrel 2, that is to say from the inside out or proximal to distal in this outlet end.

The barrel seal 3 comprises a first cylindrical wall 3.1 which houses a first cavity 3.2 and a first edge 3.3 which defines a proximal opening 3.4. The first cylindrical wall transitions into an inwardly directed horizontal plane 3.5 whose inner end forms a second cylindrical wall 3.6 and houses a second cavity 3.7. These cylindrical walls together have an L-shaped cross-section. The second cylindrical wall 3.6 transitions into a peripheral circular protruding thin tongue 3.8 (which is thinner and thus more flexible than the rest of the barrel seal) which distally has a second edge 3.9, which forms a distal mouth 3.10.

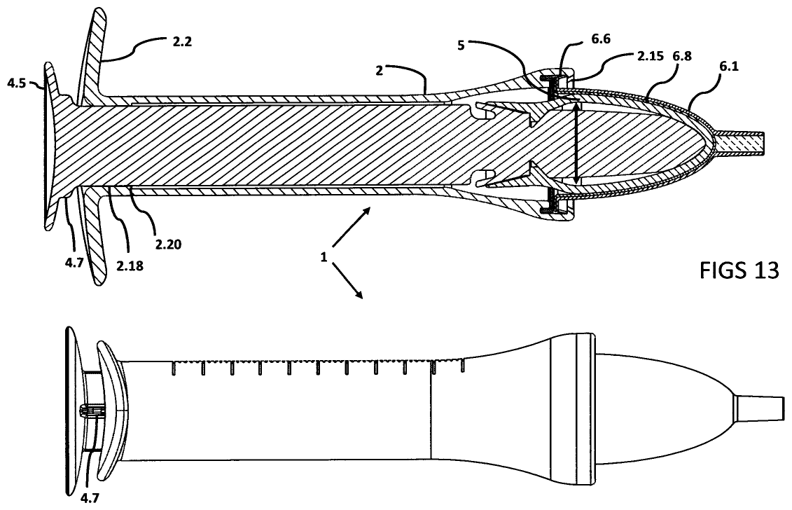

The barrel seal 3 is mounted in the distal end of the barrel in the second cylindrical cavity, in particular in the groove 2.12 as shown in FIGS. 13, where the first cylindrical wall 3.1 rests in the groove 2.12 and the inwardly directed horizontal plane 3.5 rests against the inner circular edge 2.11.

The barrel seal is suitably made of elastic material, such as elastic rubber or plastic material, e.g., silicone or nitrile.

### Plunger

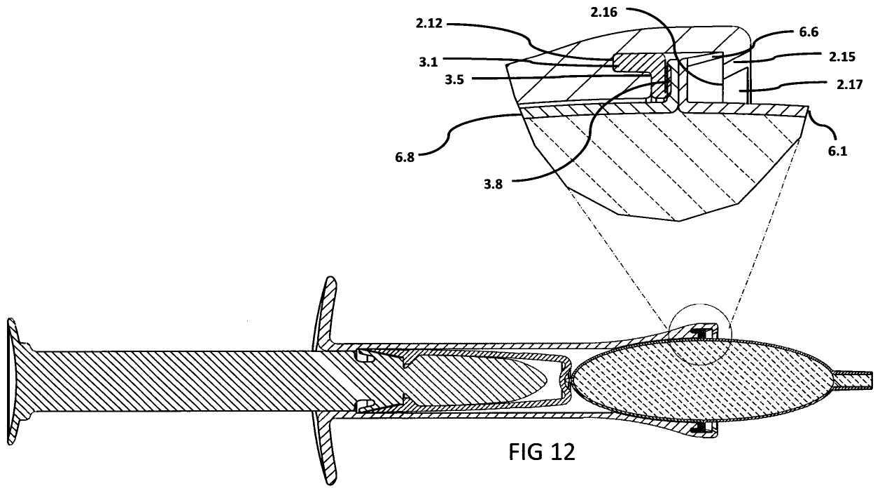

In FIGS. 4A, a plunger 4 is shown which includes a plunger rod 4.1 (essentially the plunger minus a grip part), which is accommodated in the cylindrical channel and is displaceable therein. The cylindrical cavity 2.4 in the barrel is arranged for the plunger rod to be able to move in the barrel with low friction and for air in front of the plunger head to be able to move from the conical cavity that the cone-shaped flare 2.6 encloses and the surrounding atmosphere outside the proximal mouth 2.5. The low plunger friction is a big difference to the syringes on the market that have two rubber seals that lead to high friction in both directions of movement and make them difficult to control. This high friction also becomes even higher when the syringes have been stored for a couple of months. The plunger rod 4.1 comprises a plunger body 4.2 and a plunger head 4.3. The plunger body has a grip portion 4.5 at its proximal end 4.4. The plunger rod 4.1 can have many designs, for example be solid, cylindrical or consist of joined ribs. In FIGS. 4A, an embodiment is shown where the plunger body 4.2 consists of four axial ribs 4.6 which are axially joined to a cross-like structure, in this case at a 90 degree angle, but other angles are possible. The ribs allow easy access to the barrel when cleaning the syringe when the plunger remains in it. Proximally, the plunger or ribs are joined to the grip portion 4.5, shown here as disc-shaped. Two of the ribs are provided proximally with a stop heel 4.7 which prevents the cylindrical channel from being clogged proximally when the plunger is maximally depressed. This is so that the syringe can stand upside down in a dish rack or cleaning device and cleaning liquid can pass straight through the syringe. Distally, the ribs 4.6 merge into a sloping part 4.8 which ends distally with a disc 4.9 which constitutes a circular wall, so that the cross section of the plunger body 4.2 here has the shape of a truncated cone. The disc 4.9 connects the ribs 4.6. The starting part of the sloping part is proximally slightly higher than the rest of the ribs, and below this starting part, there is a recess 4.10, which means that the proximal part of the sloping part 4.8 forms a shank 4.11, which lies slightly above the other part of the rib. At least two, and preferably two, of the ribs are provided with these shanks. When moving the plunger back, the shanks get stuck on the heel 2.20 in the barrel cavity 2.4, which prevents the plunger from slipping out of the barrel when using the syringe, i.e., back movement of the plunger (see FIG. 12). The shanks usually do not abut against the first cylindrical cavity 2.4, but do so when the plunger does not move axially in them or when negative pressure is created in the plunger when it moves back when absorbing liquid. When fitting the plunger into the barrel from its proximal end in the third barrel cavity 2.19, the shanks 4.11 will be pushed in, but then spring out when they have reached the first barrel cavity 2.4. The plunger head 4.3 essentially has the shape of an elongated barrel and can be solid or hollow. The plunger head is proximally attached to the disc 4.9 and, however, proximally has the shape of an outwardly directed cylindrical cone 4.12, which together with the sloping portion 4.8 on the plunger body forms a recess 4.13 on the plunger rod. In one embodiment, this recess 4.13 is circular. This recess forms a mount for a plunger packing described below. Distally, the cylindrical cone 4.12 merges into an elongated barrel 4.14 which terminates at its distal end with a convex, ellipsoidally arched nose portion 4.15 which is on its outer surface. Axial ribs 4.16 run over the plunger head and are joined to the nose portion (see FIG. 4A). In this embodiment, the ribs 4.6 and 4.16 lie in line with each other, but do not necessarily have to do so.

In FIGS. 4B, a plunger belonging to the second embodiment of the injection syringe 1 is shown, and which fits the embodiment of the barrel 2 shown in FIG. 2B. The plunger 4 has the same plunger head 4.3 as in the first embodiment, but differs in terms of the plunger body 4.2, which in this case has no longitudinal ribs and is essentially smooth. The plunger body 4.2 has a cylindrical wall 4.17 which houses a cylindrical cavity 4.18, intended to receive an insert, e.g. a measuring scale. In this embodiment, in the proximal end/opening of the plunger, there is an annular seat 4.19 where the cylindrical wall 4.17 meets the grip portion 4.5. This seat is intended to receive a flange on an insert, and if this insert has a measuring scale, ensure that the measuring scale sits firmly so that correct measurement can take place. The plunger body 4.2 lacks a stop heel (4.7 in the first embodiment of the plunger) in this embodiment as this plunger is intended to be completely pulled out of the barrel during cleaning.

The plunger is conveniently made of plastic material such as polycarbonate or polyamide. In the second embodiment of the injection syringe, the plunger is transparent so that an insert comprising a measuring scale is visible through it.

### Plunger Insert

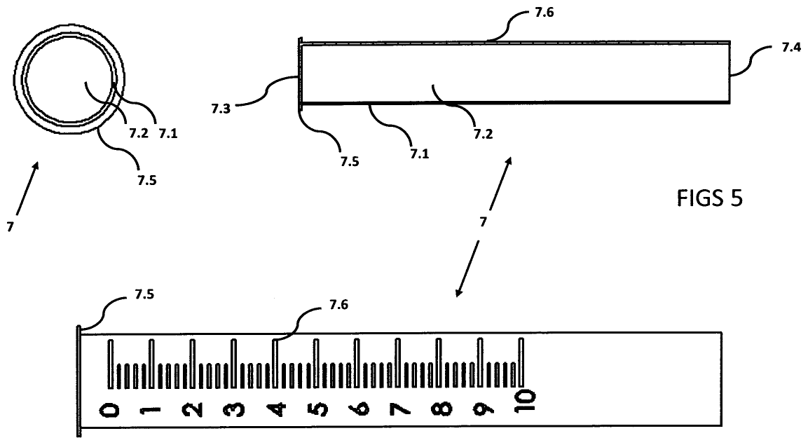

In FIGS. 5, a plunger insert 7 is shown intended for insertion into the plunger of the second embodiment of the injection syringe 1. The plunger insert 7 comprises a cylindrical wall 7.1 which houses a cavity 7.2, a proximal end 7.3 and a distal end 7.4. These ends can be open or closed. At the proximal end 7.3 there is a peripheral external flange 7.5, which at least partially runs along the cylindrical wall 7.1. When inserted into the plunger 4 (the cylindrical and hollow), this flange is received on the seat 4.19 of the plunger 4, which stabilizes the insert. The barrel set 7 can further include a measuring scale 7.6. The seat 4.19 ensures that the plunger insert sits stably and readings can take place correctly. Because the insert is protected in the plunger the measuring scale will be protected against wear, which is beneficial for repeated use.

The plunger insert insert 7 is suitably made of plastic material such as polycarbonate or polyamide, and is preferably transparent.

### Plunger Packing

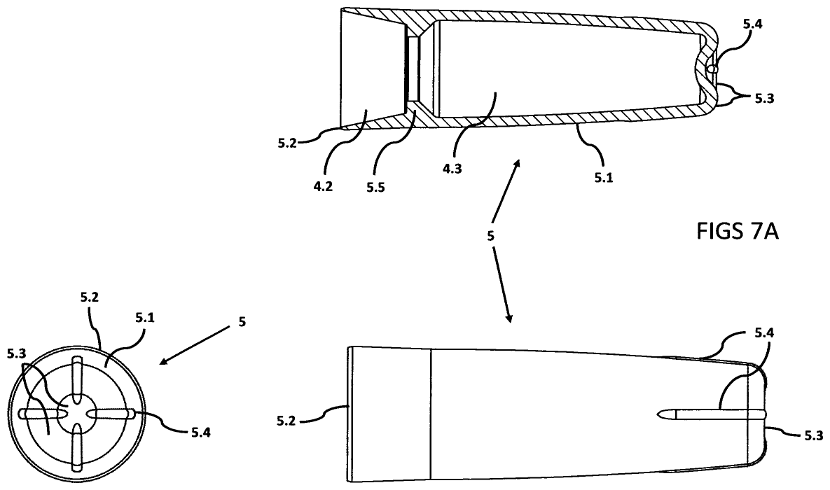

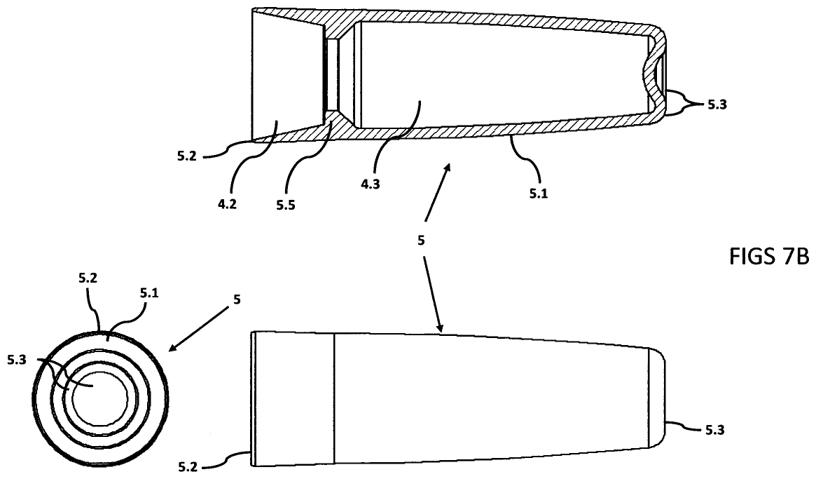

In FIGS. 7A, an elongated cover-shaped plunger packing 5 is shown, comprising a cylindrical wall 5.1 which is limited at its proximal end by a proximal edge 5.2 and distally by a end wall 5.3 which is concave ellipsoidally arched towards its interior (when unloaded) forming its distal end. On the outside, the barrel wall has distal axially extending ribs 5.4, for example four or more, which have a height of the order of 0.1 mm, and which run up to the concavity of the end wall and at least partially above it. Internally, the barrel wall has an internal flange 5.5. In one embodiment, this flange is circular. When assembling the syringe, the plunger packing is pressed over the plunger head 4.3, whereupon the flange 5.5 engages in the recess 4.13, which locks the plunger packing. The plunger packing's elastic material will then tightly enclose part of the plunger's body 4.2 as well as the long sides of the plunger head 4.3 up to its nose portion 4.15, but the plunger packing is designed so that its end wall 5.3 lies at a distance from the plunger head's nose portion 4.15 (when it is unloaded by the nose portion 4.15, this is described more below).

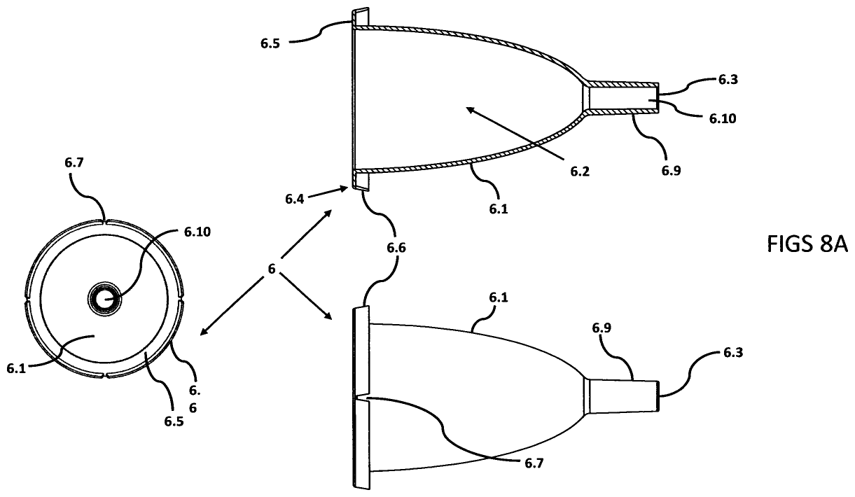

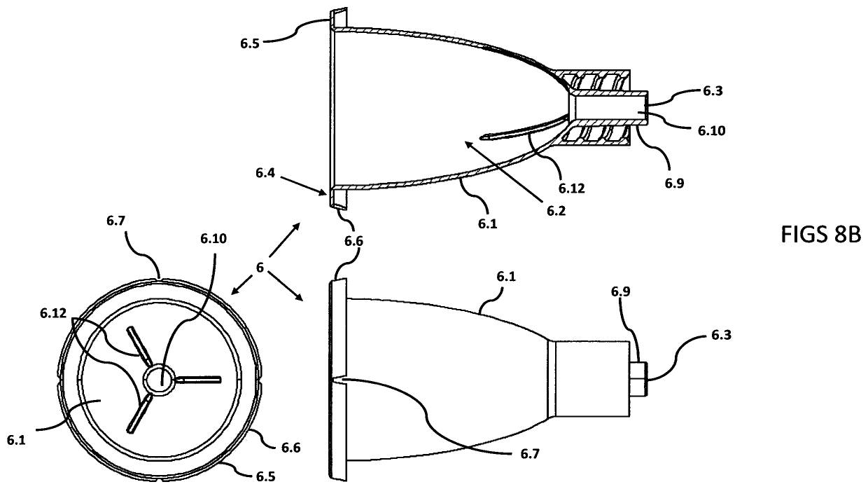

In FIGS. 7B, another embodiment of the plunger packing 5 is shown, which fits both embodiments of the plunger 4 (as shown in FIGS. 4A and 4B) and is intended for the embodiment of the capsule 6 with grooves 6.12 in the hard part 6.1 of that capsule, which e.g., is shown in FIG. 8B. The only difference between the two embodiments of the plunger packing 5 is that the barrel wall 5.1 does not have distal axially extending ribs 5.4 on the outside. These are replaced by the grooves 6.12 in the hard part 6.1 of the capsule (which are easier to manufacture from a production point of view), which have the same function and give the same effect.

The plunger packing is suitably made of elastic material, such as elastic rubber or plastic material, e.g., silicone or nitrile.

### Capsule

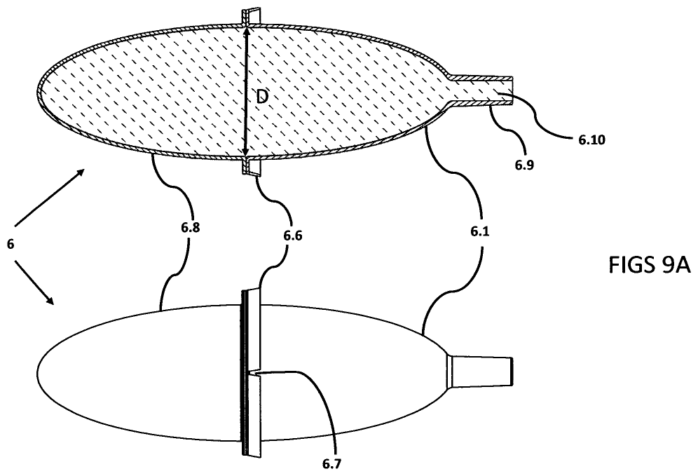

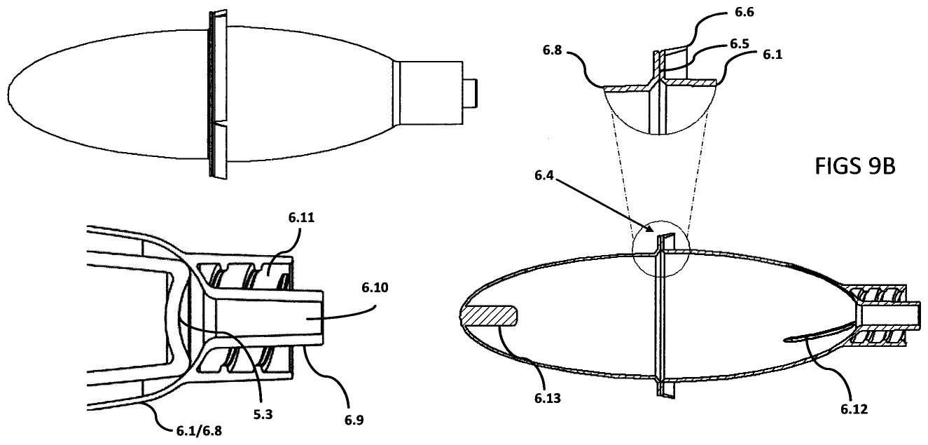

In FIGS. 8A, a capsule 6 is shown which is designed to be removably attached to the outlet end of the barrel, so that it forms the end wall of the barrel. The capsule, as it is generally referred to, comprises a mainly rigid semi-ellipsoidal first wall portion 6.1, which forms an external surface, preferably in the shape of an elongated semi-ellipsoidal dome. When the capsule is attached to the discharge end of the barrel, it will have an inside diameter D (see FIG. 9A).

This first wall portion forms a proximally projecting nozzle 6.2, which tapers conically towards the outer distal end 6.3 of the nozzle for attachment of an injection needle or cannula thereon (these are not shown).

Means for removably attaching the capsule to the outlet end of the barrel comprises a peripheral circular flange 6.4 on the capsule at the proximal end of the proximally projecting nozzle 6.2. The flange may have a V-shaped notch in the underside thereof or may transition into a conical collar on one side of the flange, which projects in the same direction as the distal end 6.3. In FIGS. 8A and 9B, an embodiment is shown where the flange comprises a circular perpendicular horizontal strip 6.5 which transitions into a circular projecting strip 6.6 which projects in the same direction as the distal end 6.3. The angle of the strip 6.6 should be greater than 90° and less than 180°, e.g., 20-30. In one embodiment, the flange 6.4 has at least two breaking indications 6.7 in its peripheral edge, (in FIG. 8A in the strip 6.7). When the injection step is complete and the capsule is released from the discharge end of the barrel, these breaking indications will cause the flange to break in one or more places, showing that the capsule is used and ready for disposal.

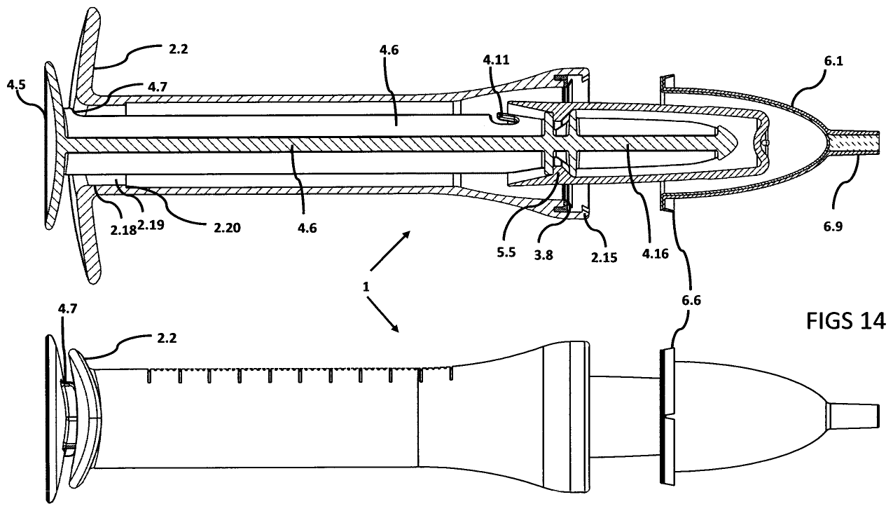

The mentioned first wall part 6.1 of the capsule 6 is preferably made of some suitable transparent plastic material, such as polypropylene or polyester, and can be manufactured by injection molding. The capsule further comprises a flexible second semi-ellipsoidal wall portion or dome 6.8 on the inside of the end wall (see FIGS. 13 and 14), which is formed by the capsule when it is attached to the barrel. The second wall part is connected to the outside of the flange 6.4, for example by melting/heat welding and welding, for example ultrasonic welding.

The wall part 6.8 is preferably made of a suitable flexible transparent plastic material. In a preferred embodiment, the wall portion 6.8 comprises a transparent foil of, for example, thermoplastic, polyamide and/or polypropylene. In one embodiment, this is a deep-drawn transparent foil laminate of polypropylene and polyamide. In another embodiment, this is an injection-molded and softened polypropylene, for example from Mediprene® thermoplastic. In one embodiment, a semi-ellipsoidal dome, which forms the inner wall portion of the capsule 6.8. It is compressible against the inner surface of the capsule wall portion 6.1 to a compressed position, as shown for example in FIG. 14, and in this position the capsule wall portion 6.8 is shown to be so dimensioned and shaped as to touch the concave inner surface of the empty capsule wall portion 6.1, whereby it follows the curvature of this wall section. This is the condition in which a non-prefilled capsule can be delivered. Capsule 6 is connected to the barrel 2 through the capsule's nozzle 6.2 and its flange 6.4 is pressed against the distal end of the barrel to accommodate the strips 2.15 on the outer circular edge 2.13. As the capsule is pushed over the strips 2.15, the flange 6.4 flexes or yields elastically as it slides against the conical guide surface 2.17 on the strips, and then springs out by its elasticity, having slid over the strips, to engage at its edge with the inner shoulder 2.16 on each strip 2.15. Through the thus obtained engagement between the barrel and the capsule, the capsule is retained by the barrel, as shown in FIGS. 12.

Referring to FIG. 9B there is shown an axial cross-section of the nozzle distal end 6.3 as shown in FIG. 1A where this end comprises a cylindrical wall 6.9 defining a distal mouth 6.10. In FIGS. 8B and 9B, a threaded portion 6.11 with a female thread is also shown on the cylindrical wall 6.9. The distal end of the nozzle may be adapted for attachment of the cannula in any of the ways common in the art. In one embodiment, this has a luer lock or luer slip fitting. In the case of a luer slip fitting (without the threaded portion 6.11), a cannula with a matching fitting is pushed over the cylindrical wall 6.9 into frictional engagement with it (FIG. 8A). In the case of a luer lock connection, the outer distal end 6.3 of the nozzle is provided with the threaded portion 6.11, into which a cannula with a suitable socket and male thread is screwed (FIGS. 8B and 9B).

The capsule may have a centered or eccentric distal mouth 6.10 (into which the cannula is attached), but it is preferably centered. Because the capsule 6 is ellipsoid, one has access to inject in all the injection angles that a normal syringe, more spherical syringe, cannot access as the barrel or capsule is obstructed by the patient's body tissues. The present injection syringe thus has the advantage that the cannula can be used for all injection angles despite the fact that the distal mouth 6.10 and the cannula attached therein is centered on the capsule.

In FIGS. 8B, another embodiment of the capsule 6 is shown which is intended for use with the smooth plunger packing (without external distal axially extending ribs 5.4, as shown in FIGS. 7B), which capsule has grooves 6.12 in the hard part 6.1 of the capsule, which has the same function as the ribs 5.4.

In FIGS. 9B, an embodiment of the weld joint is shown enlarged. Here, the wall of the capsule has been preformed into the shape of a V just before its joining point. This facilitates when the joint is to be welded together, but also when the softer wall part 6.8 is to be folded into the harder wall part 6.8 as the joint takes on a hinge-like shape and property and folds in more easily. This shape of the weld joint works with all capsules according to the invention.

FIG. 9B also shows an embodiment of the capsule 6 where the softer wall portion 6.8 is provided with an internal peg 6.13 at its bottom. When injecting the contents of the capsule, the peg will force the liquid into the nozzle of the capsule, whereby all or substantially all of the liquid is injected, which is particularly advantageous if the injected liquid is costly. The material thickness of the peg is advantageously thicker than the rest of the softer wall part 6.8. The diameter of the peg is slightly smaller than the inner diameter of the nozzle of the capsule. The peg works with all capsules according to the invention, and can be manufactured in the same material as the softer wall part of the capsule.

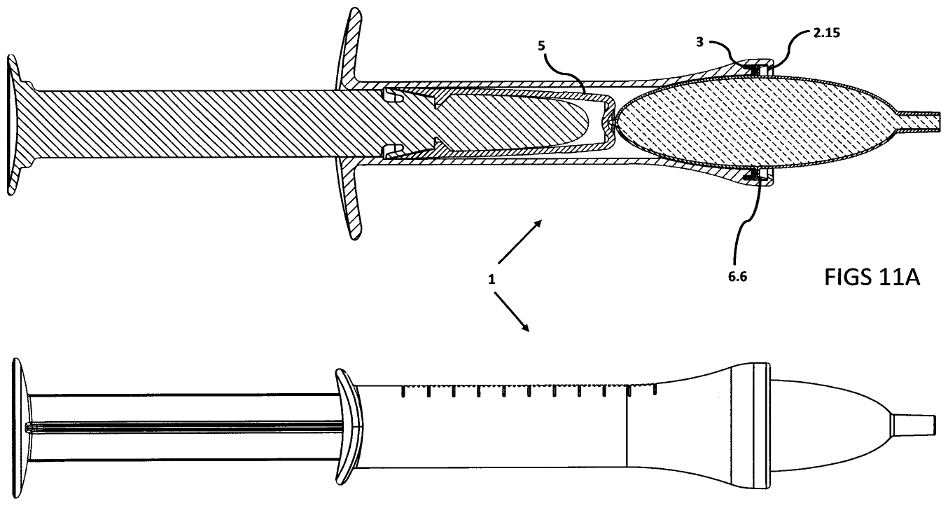

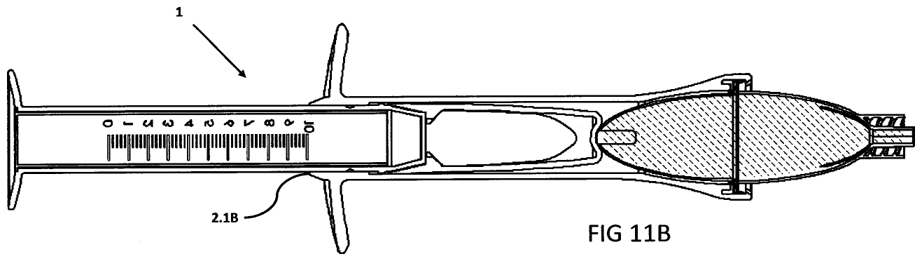

When the capsule 6 is completely filled with liquid, as shown in e.g. FIGS. 11A, it has an ellipsoidal shape, its internal diameter being larger than the internal diameter of the first cylindrical cavity 2.4. However, the semi-ellipsoidal cavity 2.8 in the barrel 2 fits the semi-ellipsoidal shape of the filled capsule, which is partially occupied therein, as shown in FIG. 11A and FIG. 12. The tongue 3.8 of the barrel seal 3 seals the outlet end of the barrel by abutting against the capsule's 2 circular flange 6.4 when a filled or empty capsule is attached to the barrel. The tongue 3.8 seals against the capsule when negative pressure occurs in the syringe when the plunger is pulled back, and releases air when the plunger is pushed forward during injection and overpressure is created via the breaking indications 6.7 on the capsule. The sealing tongue 3.8 has proven to make the syringe more robust in use, as it takes care of small irregularities in the capsule wall that lead to air being able to enter the barrel chamber of the syringe when an unfilled capsule is to be filled and the plunger is pulled back. Air in the capsule leads to incorrect determination of the liquid contained in it and thus the amount intended to be injected, which is why the suction step must be redone.

The capsule 6 can also be supplied as a pre-filled capsule which is closed, for example with a foil at its distal end. When the capsule is attached to the barrel 2, the cannula is mounted on the nozzle as described above. The cannula can also have a bidirectional needle, where one part sticks into the cannula socket, whereupon when mounted on the pre-filled capsule punctures the foil and creates a free flow path through the needle of the cannula. The filled capsule can also be closed with a cap (e.g. via luer lock or luer slip), where the cap is removed and the cannula is mounted on the distal end of the capsule.

Liquid is expelled from the capsule in the manner described above starting from the moments shown in FIGS. 11A to 13.

From the above description of the various parts of the syringe and the capsule, it appears that the plunger 3 has a convex, ellipsoidal arched nose portion 4.15 which corresponds to the ellipsoidal shape of the capsule. Furthermore, in the preferred embodiment of the injection syringe according to the invention, as described herein, the radius of curvature of the nose portion 4.15 of the plunger head 4.3 (and the end wall 5.3 of the plunger packing) is substantially the same as the radius of curvature of the inside of the flexible wall portion 6.8 of the capsule as it is pressed against the inner the surface of the wall part 6.1 of the capsule and closely follows the shape thereof, as shown in FIG. 13. The ellipsoidal concave surface 5.3 of the plunger packing has essentially the same radius of curvature as the outer surface of the wall part 6.8 of the capsule 6, when the capsule is completely filled with liquid, as shown in FIGS. 11A, where the filled capsule is generally ellipsoidal in shape. The radius of curvature of the convex nose portion 4.15 of the plunger is equal to the radius of curvature of the capsule wall portion 6.8, as it follows the shape of the capsule wall portion 6.1 in the depressed state, minus the wall thickness of the end wall 5.3 of the plunger packing. The flexible end wall 5.3 of the plunger packing has, due to the pressure exerted on it by the nose portion of the plunger head at this stage, gone from being concavely curved to being convexly curved. At the same time, the long sides of the plunger packing have expanded to rest against the flexible wall portion 6.8 of the capsule. The length of the plunger packing is designed (when mounted on the plunger, unloaded and non-deformed, see FIG. 11A) so that the distance between the distal end of the plunger packing (plunger packing end wall 5.3) and the plunger nose portion 4.15 is such that the plunger packing in the final stage of injection (see FIG. 14) pushes out the wall part of the capsule 6.8 all the way towards the wall part of the capsule 6.1) so that all the liquid between the wall parts 6.8 and 6.1 is injected. The expansion of the plunger packing towards the capsule wall portion 6.8 takes place essentially from the point where the circular flange 5.5 of the plunger packing engages in the recess 4.10 of the plunger.

The guide of the plunger rests against the inner cylindrical surface of the third cylindrical wall 2.18, and is self-centered in the barrel by the shanks 4.11 of the plunger packing. This applies before plunger packing deformation, which is described more below. In FIGS. 14, the plunger rod 4.1 is drawn a little narrower (does not abut against the third barrel cavity 2.19 at all). This is only done so that you can more easily see the stop heel or surface 2.20. One of the shanks 4.11 is also not drawn in this figure.

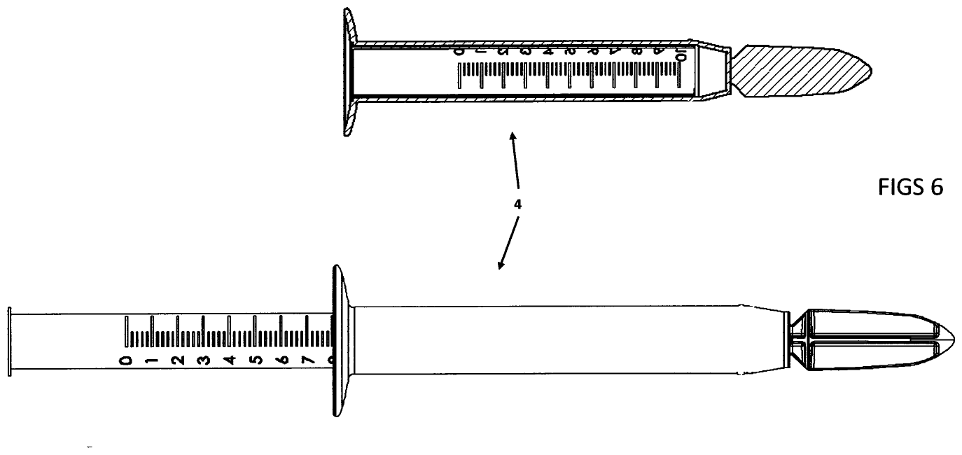

The outer side of the first cylindrical wall 2.3 and the plunger rod are designed with a measuring scale and scale line which, when mounting the plunger in the barrel, match each other. The scale bars correlate with the volume contained in the capsule. Through the long narrow ellipsoidal appearance of the capsule, and the long narrow shape of the first cylindrical cavity 2.4 of the barrel 2, you get a longer stroke which means that the scale lines can be placed further apart. This makes it easier to dose the correct amount. This becomes particularly important when the difference in injected amount between two or more occasions must be very small, and when the injected amount itself is very small and an incorrectly dosed volume can give very large margins of error. In a second embodiment of the injection syringe, where the plunger 4 is hollow, cylindrical and transparent (FIG. 4B) and has an insert 7 with a measuring scale (FIG. 5), the insert is inserted with the distal end into the proximal end of the plunger until its flange 7.5 is received in the annular seat 4.19 of the plunger 4 (FIG. 6). This plunger and insert mates with the second embodiment of the barrel (FIG. 2B). When the plunger 4 and the insert 7 move in the barrel 2, the measuring scale on the insert can be read against the matte/opaque/less transparent measuring edge 2.1B on the barrel.

The long narrow ellipsoidal appearance of the capsule also provides another very important advantage, namely that the stroke length of the plunger is extended in the moment after the plunger/plunger packing has moved forward and reached the capsule wall. The pressure applied to the capsule end, which causes the plunger packing to expand and which in turn causes the capsule flange 6.4 to lose its engagement with the strips 2.15 on the outlet end of the barrel, builds up over a longer distance as the plunger needs to move further in order for the capsule diameter at the flange 6.4 to shrink enough to lose its engagement with the strips 2.15. Up to this point, the plunger and plunger packing have moved essentially friction-free in the barrel chamber. The plunger's longer stroke means that suction and injection take place over a longer distance. This also provides longer distances between the scale lines, which facilitates correct reading during both absorption and injection. Furthermore, the design of the peripheral circular flange 6.4 of the capsule, which includes a circular perpendicular horizontal strip 6.5 which transitions into a circular projecting strip 6.6 (FIG. 9B), means that a significantly greater force is required when releasing the capsule than when injecting. The reason for this is that the plunger construction means that it runs almost friction-free in the barrel, but when releasing the capsule it is required that the strip 6.6 must be bent backwards to get out of engagement with the inwardly directed strips 2.15 of the barrel 2. This means that the user can easily feel the difference between the functional mode which means completed injection and the mode when the capsule is removed from the syringe. In this way, the risk of premature release of the capsule is reduced with all the disadvantages this entails in the form of incorrect dosing, safety risks for the user and contamination. In addition, the injection takes place over a longer distance, which is why the distance between completed injection and release of the capsule is longer and thus the volume per unit length is smaller. A premature release of the capsule thus leads to less error in the injected amount.

The capsule can be delivered empty in a sterilized state, where the wall part 6.8 of the empty capsule is pressed into contact with the concave inner surface of the wall part 6.1 of the empty capsule 6, where the wall part 6.8 follows the curvature of the wall part 6.1, in the corresponding manner as in FIGS. 14. use of the injection syringe, the capsule is attached to the barrel as described and shown in FIG. 11A. If the plunger head/plunger packing does not already touch the outer surface formed by the wall portion 6.8, which is pressed against and follows the curvature of the inner surface of the wall portion 6.1 of the empty capsule, it is manually moved to such a position that it touches the wall portion 6.8, as also shown in FIG. 13. As the plunger head (including the plunger packing mounted thereon) is displaced towards the capsule at the outlet end of the barrel, which is closed by the capsule, which forms an end wall on the barrel, air trapped between the nose portion of the plunger 4.15 and the end wall 5.3 of the plunger packing, when the end wall is deformed into a convex shape and the diameter of the plunger packing expands, to pass between the plunger head and the plunger packing and be discharged proximally into the barrel chamber at the proximal edge of the plunger packing 5.2. The atmosphere communicates with the interior of the barrel through the clearances that exist between the plunger/plunger packing and the barrel chamber and the semi-ellipsoidally arched inner wall 2.7.

A cannula (not shown) is then inserted into a bottle or the like, containing a liquid medicine, which is to be taken up by the capsule, or into a blood vessel for taking a blood sample therefrom. The part of the first cylindrical cavity 2.4 which is behind the plunger head/plunger packing is ventilated through the clearance between the ribs 4.6 of the plunger and the proximal mouth 2.5, so that no pressure builds up behind the plunger head. However, a negative pressure (partial vacuum) will occur in the first cylindrical cavity 2.4 between the plunger packing and the end wall formed at the outlet end of the barrel by the capsule 6, where a leak-proof seal is arranged between the capsule and the cone-shaped flare 2.6 through the barrel seal 3. No air will able to pass into this space from the surroundings, since effective sealing is also maintained between the plunger packing and the inner surface of the barrel as the plunger is withdrawn from the outlet end, as explained above. Through the negative pressure thus created, the wall part 6.8 will be successively pulled away from the wall part 6.1 to the position shown in FIG. 13, and the capsule will be filled with liquid.

To eject the liquid from the filled capsule in FIGS. 11A, for example for injecting a drug or for transferring a blood sample to a test tube, the capsule is emptied by pressing the plunger/plunger packing against the filled capsule. During this operation, the cannula needle is inserted into a blood vessel or into the tissues of a human or animal for injection of the drug or alternatively into a test tube or other capsule, to which the blood sample is to be transferred for treatment or test operations.

During displacement of the plunger/plunger packing axially towards the capsule 6 from the position in FIGS. 11A, in which the ellipsoidally curved concave end wall 5.3 of the plunger packing 5 abuts against the ellipsoidally curved outer surface of the flexible capsule wall portion 6.8, this wall portion will be pushed in mechanically by the plunger/plunger packing and will be pressed against the inner surface of the capsule wall portion 6.1, as shown in FIG. 13. Previously, it has been described that the proximal edge 5.2 of the plunger packing releases excess pressure in the syringe created by the forward movement of the plunger. Furthermore, it has been described that the barrel seal's sealing tongue 3.8 also releases any excess pressure via the breaking indications 6.7. This is another safety aspect of the invention. If air was allowed to pass into the capsule through a perforation in a defective capsule wall portion 6.8, the air could enter the a blood vessel, which could be dangerous for the patient and even life-threatening, or the formation of compressed air in front of the plunger head/plunger packing could result in ejection of the capsule from the conical flare 2.6. If liquid leaks into the first cylindrical cavity 2.4 of the barrel 2 from a defective capsule, it could be contaminated by contact with the barrel and plunger. Such contaminated liquid will pass to the back of the plunger head/plunger packing through the cylindrical cavity 2.4, where the flow resistance is lower than that in the cannula, and thus will not be injected from the injection syringe. This is an additional safety feature of the injection syringe according to the invention.

When the plunger head 4.3/plunger packing end wall 5.3 has just reached the capsule wall portion 6.8/6.1, the bulk of the liquid has been injected, and the distal end of the plunger packing (the ribs on it) has just reached the capsule wall portion 6.8, as it is pressed against the capsule wall portion 6.1. At this stage of operation, the plunger head/plunger packing end wall has not fully pushed the liquid out of the capsule; there is still liquid left between the capsule wall parts 6.1 and 6.8 centrally in front of the plunger packing's concave end wall 5.3 and the sides of the plunger packing (from its engagement in the plunger packing's recess 4.10). Liquid could be trapped there when the flexible wall part 6.8 is pressed against the wall part 6.1 by the plunger head 4.3/plunger packing which has a smaller diameter than the internal diameter of the capsule. However, thanks to the plunger packing's concave end wall 5.3 and the ribs 5.4, there is a free flow path up to the capsule's distal mouth 6.10 and essentially no liquid is trapped.

In the next functional step for expelling the liquid from the capsule 6, the plunger rod/plunger packing is further displaced axially, whereupon the pressure on the plunger packing increases in the area where the circular flange 5.5 of the plunger packing engages with the recess 4.10 on the plunger so that the side wall portion of the cylindrical wall 5.1 of the plunger packing will be deformed to bulge (and its diameter increase, see the double-directional arrow in FIG. 13) and is pressed against the depressed capsule wall portion 6.8 in the region thereof, which is located distally adjacent to the flange 6.4. Furthermore, the convex ellipsoidally arched nose portion 4.15 of the plunger head 4.3 will abut against the inner surface of the ellipsoidally arched concave end wall 5.3 of the plunger packing and deform this portion, so that it is made to follow the ellipsoidal shape of the depressed capsule wall portion 6.8 (the end wall 5.3 goes from concave to convex), as shown in FIG. 13. The thus deformed end wall 5.3 of the plunger packing will then closely rest against the wall part 6.8, which is transferred to a fully pressed position, whereupon this end part follows the curvature of the inner surface of the wall part 6.1. The ribs 5.4 will, however, ensure a free flow path up to the capsule's distal mouth 6.10 and that no liquid is trapped. Consequently, at the end of this step, no pockets will remain between the wall portions 6.1 and 6.8, and the entire amount of liquid originally contained by the capsule will have been expelled therefrom.

In an embodiment of the plunger packing, as also described above, the ellipsoidally curved outer end wall 5.3 is provided with four or more mainly radially extending thin ribs 5.4 (FIG. 7A). These strips prevent the end wall from fully pressing the capsule wall portion 6.8 against the capsule wall portion 6.1 in the functional step shown in FIG. 13 and in the previous functional step, so that liquid contained at the flange 6.4 is allowed to pass between the capsule wall portions to the distal mouth 6.10 formed by the nozzle 6.2. In an alternative embodiment of the plunger packing, as shown in FIGS. 7B, the plunger packing is smooth (without the ribs 5.4) but the capsule 6 instead has grooves 6.12 (FIG. 8B). These grooves provide the same effect as the ribs 5.4 in the first embodiment of the plunger packing.

When the liquid in the capsule has been expelled, as described above, the emptied capsule can be ejected from the barrel by means of the plunger. An increased pressure is manually applied to the deflated capsule to produce a mechanical pressure thereon at its distal end, so that the diameter of the capsule increases further distal to the flange 6.4, but where the diameter of the flange 6.4 itself is slightly reduced. Under the force thus exerted, the capsule flange 6.4 is successively pulled away from the strips 2.15 and is finally made to yield to disengage with the strips (FIG. 14). In this way, the capsule can be released from the barrel together with the needle over a waste basket and can be discarded without the need to manually touch the capsule or the needle, both of which may be contaminated with the injected fluid or by the patient. It thus appears that only a small part of the injection syringe as a whole is thrown away after each use and that this can be done in a way whereby the risk of infection spreading is avoided.

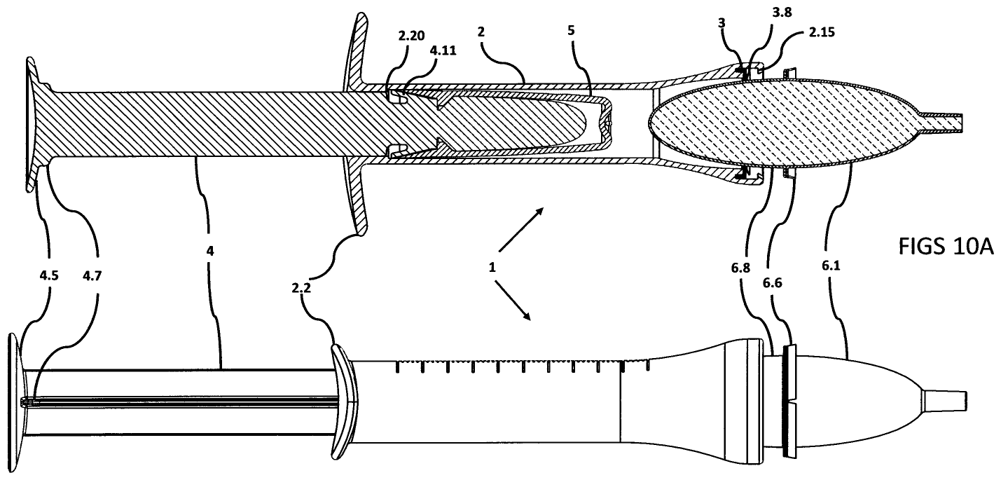

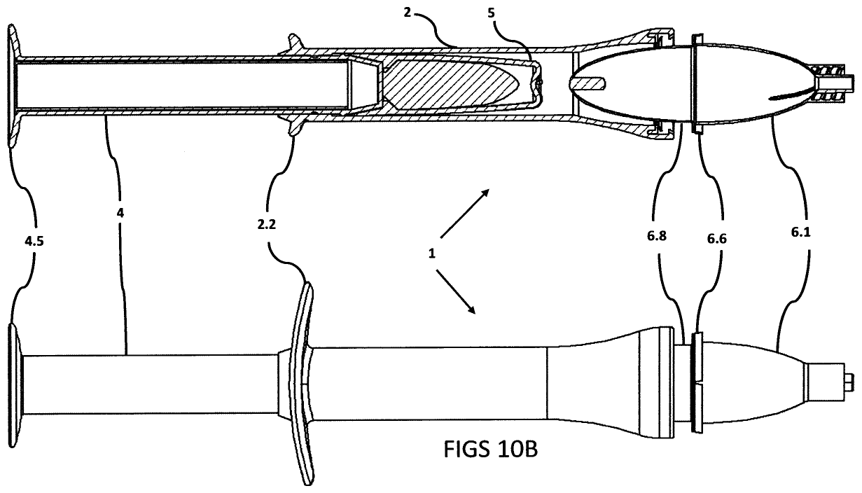

The function and operation of the alternative embodiment of the injection syringe, described more specifically in FIGS. 2B, 4B, 5 and 6, is shown in FIGS. 10B and 11B. The function and handling are essentially the same as for the first embodiment. For the purposes of the invention, the internal diameter of the barrel chamber in the barrel and the internal diameter of the capsule are related to each other in such a way that the stroke volume of the plunger between the position when an empty capsule is mounted on the syringe and the position when the plunger is withdrawn and the capsule is filled is essentially equal to the volume of the capsule.

It is assumed that the inner diameter of the barrel's first barrel cavity 6.4 is d, the inner diameter of the capsule D and the stroke of the plunger when the empty capsule is mounted to the position when the capsule is full is L. The stroke volume of the plunger is determined by the relationship:

$${\frac{π d}{4}}^{2} \times L \qquad ( 1 )$$

and the volume of the ellipsoidal capsule is determined by the relation

$${\frac{π D}{6}}^{3} \qquad ( 2 )$$

In order for the flexible wall part 6.8 to closely follow the movement of the plunger packing nose portion 4.15 during the filling operation, the stroke volume for a stroke length L, which is equal to D, must be equal to the volume of the capsule (so that the plunger packing expands correctly in the final stage of the injection and pushes out all the liquid). It follows from (1) and (2) that

$${\frac{D π d}{4}}^{2} = {\frac{π D}{6}}^{3} \qquad ( 3 )$$

$$D = \sqrt[D]{\frac{2}{3}} \qquad ( 4 )$$

$$d ≅ D \times 0 , 82 \qquad ( 5 )$$

Thus, in the preferred embodiment, the internal diameter of the barrel cavity formed by the barrel should be approximately 82% of the internal diameter of the ellipsoidal capsule. Furthermore, the ratio between the inner diameter D of the rigid wall part 6.1 of the capsule and the inner diameter (d) of the cylindrical cavity 2.4 must be approximately 1:√(⅔) in order for the flexible wall part 6.8 to have contact with the end wall 5.3 of the plunger packing during the filling of the capsule.

The hardness of the material from which the plunger packing is made, and the dimensions of the end wall 5.3 of the plunger packing and its wall thickness should be chosen so that the end wall 5.3 of the plunger packing and the side wall portion of the cylindrical wall 5.1 of the plunger packing can be deformed against the compressed capsule 6 for the complete expulsion of the liquid therefrom in the described manner by a force not sufficient for ejection of the capsule from the barrel. Otherwise, the capsule could be ejected before the operation of injecting the liquid has been completed.

A syringe size can be used to inject different volumes (eg 2, 5 or 10 ml) using a capsule size that holds at least this volume. That is, a 10 ml syringe with a first barrel cavity 2.4 adapted for a 10 ml capsule can be used for injecting volumes less than 10 ml. If you want to inject a specific volume, e.g. 2 ml or less, with high accuracy, you should preferably choose a 2 ml syringe with barrel bore 2.4 adapted for a 2 ml capsule. If you have to inject the same volume many times, for example 2 ml, you choose a 2 ml syringe with barrel cavity 2.4 adapted for a 2 ml capsule for a simple and precise injection procedure.

The semi-ellipsoidal shape of the capsule and matching semi-ellipsoidal parts of the injection syringe means that the capsule of the syringe is 2-4 times as long as the corresponding spherical capsule, i.e., the ratio between length and width of the capsule is up to 4 times. The length of the capsule can in various embodiments be 2.5-3.5 or 3-3.5. The corresponding ratio applies to matching semi-ellipsoidal parts of the injection syringe, whereby the stroke of the plunger is correspondingly much longer.

It should be obvious to the expert in the field that various modifications and variations can be made of the injection syringe according to the invention and the capsule that forms part thereof, without departing from the inventive idea.

1. An injection syringe (1), comprising an elongated barrel (2) with an outlet end with a substantially conical flare (2.6) with a semi-ellipsoidal inner wall (2.7), a semi-ellipsoidal capsule (6) when empty and ellipsoidal when filled, a device (2.15, 6.4) for removable attachment of the capsule to the discharge end of the barrel, to form the barrel end wall, and a plunger (4) fitted for reciprocating movement in the barrel, the capsule having an essentially rigid first portion (6.1) on the outside of the end wall, forming an external surface, a flexible second portion (6.8) on the inside of the end wall, which is compressible against the internal surface of the first section of the wall, and a nozzle (6.2) on the said first section of the wall for attaching a cannula or needle thereto, characterized by the fact that the barrel capacity of the plunger (4) between the displacement positions of the plunger, corresponding to empty and filled capsules (6), is essentially equal to the volume of the capsule, and that the plunger (4) comprises a plunger rod (4.1) with a body (4.2) and a head (4.3), and a hollow plunger packing (5) made of elastic material which, when unloaded, tightly encloses part of the body (4.2) and the long sides of the head (4.3) but not its nose portion (4.15); and an end wall (5.3) of the plunger packing is at a distance from a nose portion (4.15) on the plunger head.

2. The injection syringe according to claim 1, characterized by the capsule (6) being predominantly ellipsoidal and the barrel having a first cylindrical wall (2.3) forming a mainly first cylindrical cavity (2.4) having an internal diameter (d) smaller than an internal diameter (D) of the ellipsoidal capsule (6).

3. The injection syringe according to claim 1, characterised by a ratio between the internal diameter (D) of the rigid portion of the capsule (6.1) and the internal diameter (d) of the cylindrical cavity (2.4) of approximately 1:\sqrt{2/3} to allow the flexible wall portion (6.8) to be in contact with the end wall of the plunger packing (5.3) during the filling of the capsule.

4. The injection syringe according to claim 1, characterised in that the end wall of the plunger packing (5.3) has a concave ellipsoidal arching towards to the hollow interior of the plunger packing when unloaded.

5. The injection syringe according to claim 1, characterised by a radius of curvature of the ellipsoidally arched end wall of the plunger packing (5.3) being essentially equal to the radius of curvature of the flexible second wall portion (6.8), when pressed by the head of the plunger (4.3) against the internal surface of the first section of the wall (6.1), in which position the end wall (5.3) has progressed from a concave to a convex ellipsoidal curvature.

6. The injection syringe according to claim 1, characterised by a convex nose of the plunger (4.15) having substantially the same radius of curvature as the ellipsoidally curved end wall of the plunger packing (5.3) and the capsule wall portions (6.1) and (6.8).

7. The injection syringe according to claim 1, characterised by the difference between the radius of curvature of the nose portion of the plunger (4.15) and the radius of the flexible second wall (6.8) of the capsule, when pressed against the internal surface of the first portion (6.1) of the capsule, is essentially equal to the thickness of the end wall of the plunger packing (5.3).

8. The injection syringe according to claim 1, characterised by the plunger packing having a circular internal flange (5.5) and the plunger having a recess (4.13) which absorbs the flange for attaching the plunger packing to the plunger.

9. The injection syringe according to claim 1, characterised in that the syringe having a barrel seal (3) of elastic material for mounting in a groove (2.12) at the outlet end of the barrel, and where this seal has first and second cylindrical walls (3.1) and (3.6), respectively, which together have the shape of an L, and where the second cylindrical wall (3.6) merges into a peripheral circular protruding tongue (3.8) which is thinner and more flexible than the cylindrical walls (3.1) and (3.6).

10. The injection syringe according to claim 1, characterized in that the ellipsoidal capsule (6) lacks spherical or straight cylindrical wall areas.

11. The injection syringe according to claim 1, characterised in that the body of the plunger rod (4.2) is cylindrical, hollow and transparent.

12. The injection syringe according to claim 11, characterized in that the plunger (4) has a seat (4.19) at its proximal end for the reception of an external flange on a plunger insert.

13. The injection syringe according to claim 12, characterized in that the syringe further includes a plunger insert (7) having at its proximal end (7.3) a peripheral external flange (7.5).

14. The injection syringe according to claim 13, characterized in that the plunger insert (7) has a measuring scale (7.6).

15. The injection syringe according to claim 1, characterized in that the barrel at its proximal end has a measuring edge (2.1B) for reading a measuring scale on the syringe.

16. The injection syringe according to claim 1, characterized in that capsule has grooves (6.12) in the rigid portion (6.1) of the capsule.

17. The injection syringe according to claim 1, characterized in that the capsule at its proximal end has a peripheral circular flange (6.4) including a circular perpendicular horizontal strip (6.5) which merges into a circular projecting strip (6.6).

18. The injection syringe according to claim 1, characterized in that the capsule has an internal peg (6.13) at the bottom of the soft portion (6.8) of the capsule (6.8).

19. The injection syringe according to claim 1, characterized by the ellipsoidal capsule (6) having a length-to-width ratio of 2 to 4, or 2.5 to 3.5 or 3 to 3.5.

20. A kit containing the injection syringe defined in claim 1, including one or more parts of the syringe, and where the syringe is unassembled or wholly or partially pre-assembled.
